(19) United States
(12) Patent Application Publication
(10) Pub. No.: US 2026/0348859 A1
(43) Pub. Date: Nov. 5, 2026

(54) HIGH-SENSITIVITY GOLD-COATED FIBER BRAGG GRATING SENSOR FOR CHLORIDE IN AQUEOUS ENVIRONMENTS

(71) Applicant: University of Houston, Houston, TX (US)

(72) Inventors: Driss BENHADDOU, Pearland, TX (US); Stanko BRANKOVIC, Houston, TX (US); Summer Mariam DALGAMOUNI, Houston, TX (US)

(21) Appl. No.: 19/643,163

(22) Filed: Apr. 9, 2026

Related U.S. Application Data
(60) Provisional application No. 63/785,668, filed on Apr. 9, 2025.

(51) Int. Cl. G01N 21/77; G01L 1/24; G01L 11/02; G01N 33/18; G02B 6/02

(52) U.S. Cl. CPC G01N 21/7703; G01L 1/246; G01L 11/025; G01N 33/182; G02B 6/02104; G01N 2021/7709

(57) ABSTRACT

An optical probe for sensing a chemical species in a solution. The optical probe includes a fiber core having a plurality of Bragg grating elements, the grating elements comprise periodically spaced regions in the fiber core altered to have different refractive indexes; a cladding disposed about the fiber core; and a gold ultra-thin film coating disposed about the cladding. An apparatus and a method for sensing a chemical species in a solution are also provided.

### RELATED APPLICATIONS

This application claims the benefit of priority to U.S. Provisional Patent Application No. 63/785,668, filed Apr. 9, 2025, the content of which is incorporated herein by reference in its entirety.

### FIELD

The present disclosure relates generally to optical probes, and more particularly to a fiber optic Bragg grating sensor for detecting chloride levels.

### BACKGROUND

Detection of low levels of chloride ions (Cl−) in water or other effluents is of great importance for human health, prevention and control of environmental pollution, biochemical and chemical reactions, semiconductor processing and corrosion. Traditional approaches for chloride sensing involve a whole array of different detection techniques. They include volumetric titrations, electrochemical potentiometric and coulomb-metric analysis, photometric and spectroscopic methods, and chromatography. These detection approaches have different applications, scopes, and accuracies and, very often, require sampling of the solution volume to undergo analysis in a separate analytical unit. However, for medical therapeutics applications, there is an urgent need for the development of chloride sensors capable of accurate and instant detection of Cl⁻ ions at a very low concentration range ([Cl⁻]<10⁻⁴ M).

In particular, there is a need for end-stage renal disease (ESRD) patients undergoing dialysis who consume large quantities of pure water (>300 lit/week). An undetected amount of chloride contamination in water for dialysis poses serious health risks leading to infections as one of the leading causes of death in ESRD patients. Therefore, the development of an accurate, real-time Cl⁻ sensing concept would alleviate these risks, and enable safe implementation of portable Hemodialysis machines to treat patients at home.

Various fiber optic sensing devices are known and are in use for physical and chemical detection and measurement. As one example, Fiber Bragg Grating (FBG) sensors are employed to measure parameters such as strain, seismic vibrations, pressure, flow rate and temperature in components such as exhaust systems, nuclear reactors, combustors, and compressors. Conventional FBG sensors employed for dynamic strain and temperature measurements include a grating structure inscribed on to a photosensitive single-mode fiber core.

Accordingly, there is a need for a sensor that can be employed to detect a parameter, such as low levels of chloride ions (Cl⁻) in water.

### SUMMARY

This disclosure relates to a new approach to anion sensing using the Cl⁻ as a model species. The sensing electrode is an optical fiber with Brag Grating whose surface is coated with gold ultra-thin film. The sensing mechanism combines strain dependence of the Brag wavelength with the change in capillary pressure on the fiber induced by Cl⁻ adsorption. This work illustrates an ultra-fast, real-time sensing method which is generally applicable to any situation where chemical and/or electrochemical adsorption of targeted species occur on the FBG sensor surface.

In one aspect, provided is an optical probe for sensing a chemical species in a solution. The optical probe includes a fiber core having a plurality of Bragg grating elements, the grating elements comprise periodically spaced regions in the fiber core altered to have different refractive indexes; a cladding disposed about the fiber core; and a gold ultra-thin film coating deposited on the cladding.

In some embodiments, the cladding comprises polymethyl methacrylate (PMMA) or other materials such as silica, glass, etc.

In some embodiments, the cladding comprises a double cladding structure having a first cladding having 5 micrometers to about 20 micrometers radius of a first material and a second cladding having a radius from 20 micrometers to about 62.5 micrometers of a second material.

In some embodiments, the first and second claddings provide a waveguide structure for light propagation along the fiber core with negligible transmission loss even at elevated temperatures or in high radioactive environments.

In some embodiments, the plurality of Bragg grating elements are configured to reflect in phase wavelengths of light corresponding to a grating period of the grating elements.

In some embodiments, the sensing mechanism combines strain dependence of the Brag wavelength with the change in capillary pressure on the fiber induced by a species adsorption process.

In some embodiments, the chemical species is a chloride ion.

In another aspect, provided is an apparatus for sensing a chemical species in a solution. The apparatus includes an electrochemical cell having an elongated tubular shape for receiving a solution of interest; an optical probe positioned within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding.

In some embodiments, the apparatus further includes a first vertical port for receiving a reference electrode therethrough.

In some embodiments, the apparatus further includes a second vertical port for receiving a counter electrode therethrough.

In some embodiments, the apparatus further includes a sensing or working electrode disposed within the apparatus.

In some embodiments, the apparatus further includes a light source for providing an input broadband light signal to the optical sensor.

In some embodiments, the cladding of the optical probe comprises polymethyl methacrylate (PMMA) or other materials such as silica, glass, etc.

In some embodiments, the plurality of Bragg grating elements of the optical probe are configured to reflect in phase wavelengths of light corresponding to a grating period of the grating elements.

In some embodiments, the sensing mechanism of the optical probe combines strain dependence of the Brag wavelength with the change in capillary pressure on the fiber induced by a species adsorption process.

In some embodiments, the chemical species is a chloride ion.

In another aspect, provided is a method for sensing a chemical species in a solution. The method includes obtaining an electrochemical cell having an elongated tubular shape for receiving a solution of interest; positioning an optical probe within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding; and providing an input broadband light signal to the optical sensor.

In some embodiments, the cladding of the optical probe comprises polymethyl methacrylate (PMMA) or other materials such as silica, glass, etc.

In some embodiments, the sensing mechanism of the optical probe combines strain dependence of the Brag wavelength with the change in capillary pressure on the fiber induced by a species adsorption process.

In some embodiments, the chemical species is a chloride ion.

### BRIEF DESCRIPTION OF THE DRAWINGS

While the present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein. This disclosure covers all modifications and equivalents as defined by the appended claims. It should also be understood that the drawings are not necessarily to scale, emphasis instead being placed upon clearly illustrating principles of exemplary embodiments of the present disclosure. Moreover, certain dimensions may be exaggerated to help visually convey such principles. Further where considered appropriate, reference numerals may be repeated among the drawings to indicate corresponding or analogous elements. The following drawings are provided, wherein:

FIG. 1 is a diagrammatical illustration of an exemplary configuration of the optical probe employed in a sensing system, in accordance with an embodiment of the present disclosure

FIG. 2A is a view showing an actual electrochemical cell, in accordance with an embodiment of the present disclosure.

FIG. 2B presents a gold-coated PMMA fiber (sensing electrode) as seen using a scanning electron microscope, in accordance with an embodiment of the present disclosure.

FIG. 2C presents a schematic view of a FBG showing Bragg grating and a gold coating, in accordance with an embodiment of the present disclosure.

FIG. 2D presents a schematic view of the experimental apparatus, in accordance with an embodiment of the present disclosure.

FIG. 3A presents a Cyclic Voltammogram for Au coated FBG from solutions: 0.1 M HClO₄+X M [Cl⁻] (X=0.0; 2×10⁻⁵; 5×10⁻⁵; 1×10⁻⁴; 5×10⁻⁴; 1×10⁻³), sweep rate 20 mVs⁻¹, in accordance with an embodiment of the present disclosure.

FIG. 3B θ as a function of [Cl⁻] in the electrolyte. Adsorption peak integration is performed between 100 mV and 400 mV limits. The dotted blue line and red solid line are eq. (2) fits to the blue dots and red squares data, in accordance with an embodiment of the present disclosure.

FIG. 4A presents (Δλ_{B}) A vs. Cl⁻ concentration. The sensing voltage is: (A) 200 mV, 400 mV and 600 mV. The insets show a zoom-in of the data for concentrations <10⁻⁴ M. Dashed and solid lines are fits of eq (16) and eq (17) to the experimental data. The values of the equilibrium adsorption constants obtained from fits are shown in the graphs, in accordance with an embodiment of the present disclosure.

FIG. 4B also presents (Δλ_{B}) A vs. Cl⁻ concentration. The sensing voltage is: (A) 200 mV, 400 mV and 600 mV. The insets show a zoom-in of the data for concentrations <10⁻⁴ M. Dashed and solid lines are fits of eq (16) and eq (17) (see below) to the experimental data. The values of the equilibrium adsorption constants are again obtained from fits are shown in the graphs, in accordance with an embodiment of the present disclosure.

FIG. 5A presents-AG vs. E (left axis) and B vs. E (right axis), in accordance with an embodiment of the present disclosure.

FIG. 5B presents (Δλ_{B}) A vs. E for [C⁻]=const., in accordance with an embodiment of the present disclosure.

FIG. 6 shows the effect of electrocapillarity on wavelength, according to an embodiment of the present disclosure.

### DETAILED DESCRIPTION

### Terminology

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

A/an: The articles “a” and “an” as used herein mean one or more when applied to any feature in embodiments and implementations of the present invention described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Above/below: In the following description of the representative embodiments of the invention, directional terms, such as “above”, “below”, “upper”, “lower”, etc., are used for convenience in referring to the accompanying drawings. In general, “above”, “upper”, “upward” and similar terms refer to a direction toward the earth's surface along a wellbore, and “below”, “lower”, “downward” and similar terms refer to a direction away from the earth's surface along the wellbore. Continuing with the example of relative directions in a wellbore, “upper” and “lower” may also refer to relative positions along the longitudinal dimension of a wellbore rather than relative to the surface, such as in describing both vertical and horizontal wells.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”.

Any: The adjective “any” means one, some, or all indiscriminately of whatever quantity.

At least: As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements). The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Based on: “Based on” does not mean “based only on”, unless expressly specified otherwise. In other words, the phrase “based on” describes both “based only on,” “based at least on,” and “based at least in part on.”

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Couple: Any use of any form of the terms “connect”, “engage”, “couple”, “attach”, or any other term describing an interaction between elements is not meant to limit the interaction to direct interaction between the elements and may also include indirect interaction between the elements described.

Determining: “Determining” encompasses a wide variety of actions and therefore “determining” can include calculating, computing, processing, deriving, investigating, looking up (e.g., looking up in a table, a database, or another data structure), ascertaining and the like. Also, “determining” can include receiving (e.g., receiving information), accessing (e.g., accessing data in a memory) and the like. Also, “determining” can include resolving, selecting, choosing, establishing and the like.

Embodiments: Reference throughout the specification to “one embodiment,” “an embodiment,” “some embodiments,” “one aspect,” “an aspect,” “some aspects,” “some implementations,” “one implementation,” “an implementation,” or similar construction means that a particular component, feature, structure, method, or characteristic described in connection with the embodiment, aspect, or implementation is included in at least one embodiment and/or implementation of the claimed subject matter. Thus, the appearance of the phrases “in one embodiment” or “in an embodiment” or “in some embodiments” (or “aspects” or “implementations”) in various places throughout the specification are not necessarily all referring to the same embodiment and/or implementation. Furthermore, the particular features, structures, methods, or characteristics may be combined in any suitable manner in one or more embodiments or implementations.

Exemplary: “Exemplary” is used exclusively herein to mean “serving as an example, instance, or illustration.” Any embodiment described herein as “exemplary” is not necessarily to be construed as preferred or advantageous over other embodiments.

Flow diagram: Exemplary methods may be better appreciated with reference to flow diagrams or flow charts. While for purposes of simplicity of explanation, the illustrated methods are shown and described as a series of blocks, it is to be appreciated that the methods are not limited by the order of the blocks, as in different embodiments some blocks may occur in different orders and/or concurrently with other blocks from that shown and described. Moreover, less than all the illustrated blocks may be required to implement an exemplary method. In some examples, blocks may be combined, may be separated into multiple components, may employ additional blocks, and so on. In some examples, blocks may be implemented in logic. In other examples, processing blocks may represent functions and/or actions performed by functionally equivalent circuits (e.g., an analog circuit, a digital signal processor circuit, an application specific integrated circuit (ASIC)), or other logic device. Blocks may represent executable instructions that cause a computer, processor, and/or logic device to respond, to perform an action(s), to change states, and/or to make decisions. While the figures illustrate various actions occurring in serial, it is to be appreciated that in some examples various actions could occur concurrently, substantially in series, and/or at substantially different points in time. In some examples, methods may be implemented as processor executable instructions. Thus, a machine-readable medium may store executable processor instructions that if executed by a machine (e.g., processor) cause the machine to perform a method.

May: Note that the word “may” is used throughout this application in a permissive sense (i.e., having the potential to, being able to), not a mandatory sense (i.e., must).

Operatively connected and/or coupled: Operatively connected and/or coupled means directly or indirectly connected for transmitting or conducting information, force, energy, or matter.

Optimizing: The terms “optimal,” “optimizing,” “optimize,” “optimality,” “optimization” (as well as derivatives and other forms of those terms and linguistically related words and phrases), as used herein, are not intended to be limiting in the sense of requiring the present disclosure to find the best solution or to make the best decision. Although a mathematically optimal solution may in fact arrive at the best of all mathematically available possibilities, real-world embodiments of optimization routines, methods, models, and processes may work towards such a goal without ever actually achieving perfection. Accordingly, one of ordinary skill in the art having benefit of the present disclosure will appreciate that these terms, in the context of the scope of the present disclosure, are more general. The terms may describe one or more of: 1) working towards a solution which may be the best available solution, a preferred solution, or a solution that offers a specific benefit within a range of constraints; 2) continually improving; 3) refining; 4) searching for a high point or a maximum for an objective; 5) processing to reduce a penalty function; 6) seeking to maximize one or more factors in light of competing and/or cooperative interests in maximizing, minimizing, or otherwise controlling one or more other factors, etc.

Order of steps: It should also be understood that, unless clearly indicated to the contrary, in any methods claimed herein that include more than one step or act, the order of the steps or acts of the method is not necessarily limited to the order in which the steps or acts of the method are recited.

Ranges: Concentrations, dimensions, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds).

As used herein, the term “sensor” includes any electrical sensing device or gauge. The sensor may be capable of monitoring or detecting pressure, temperature, displacement, vibration, resistivity, or other data. Alternatively, the sensor may be a position sensor.

### DESCRIPTION

Specific forms will now be described further by way of example. While the following examples demonstrate certain forms of the subject matter disclosed herein, they are not to be interpreted as limiting the scope thereof, but rather as contributing to a complete description.

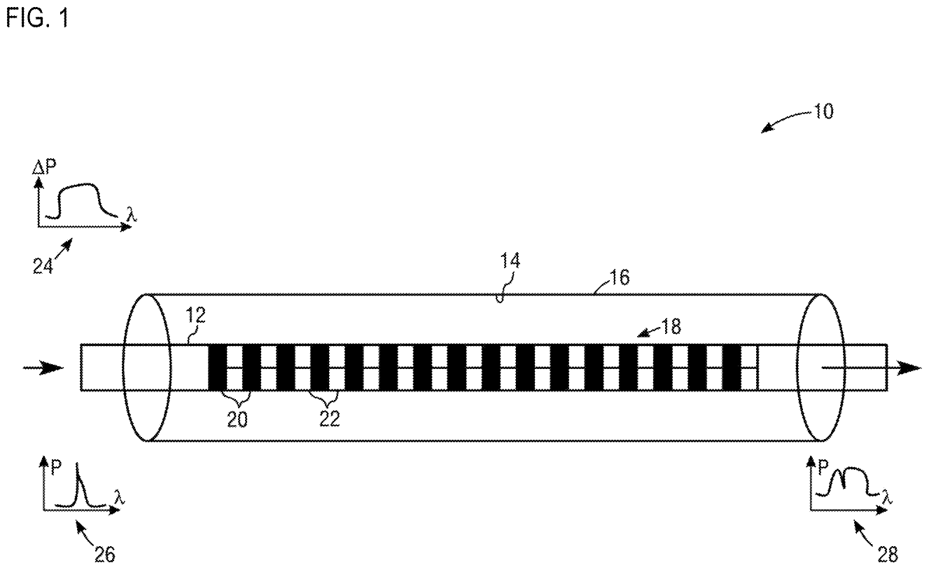

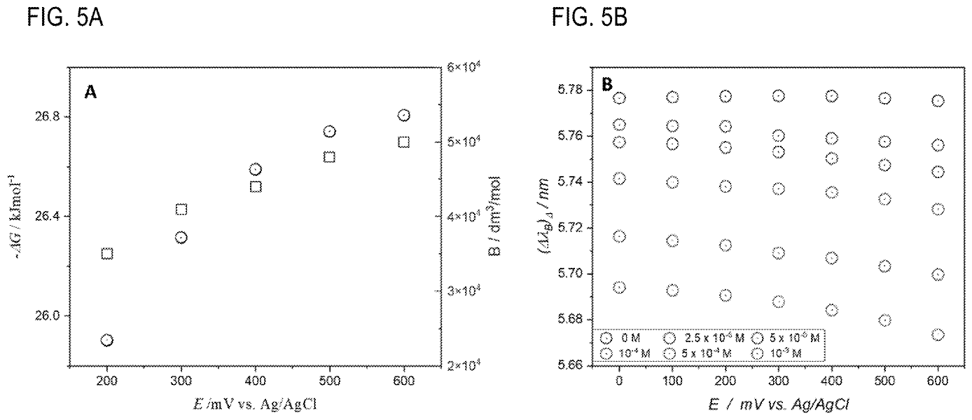

FIGS. 1-6 provide illustrative, non-exclusive examples of an apparatus, method, and system for the measurement of chloride in solution, according to the present disclosure, together with elements that may include, be associated with, be operatively attached to, and/or utilize such an apparatus, method, or system.

In FIGS. 1-6, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

In general, structures and/or features that are, or are likely to be, included in a given embodiment are indicated in solid lines in the figures, while optional structures and/or features are indicated in broken lines. However, a given embodiment is not required to include all structures and/or features that are illustrated in solid lines therein, and any suitable number of such structures and/or features may be omitted from a given embodiment without departing from the scope of the present disclosure.

Although the approach disclosed herein can be applied to a variety of apparatus designs and methods, the present description will primarily be directed to an apparatus and method for the measurement of chlorine in water.

Referring now to FIG. 1, a diagrammatical illustration of an exemplary configuration of an optical probe 10 for the measurement of a chloride in solution is depicted, in accordance with an embodiment of the present disclosure. The optical probe 10 includes fiber core 12, a cladding 14 disposed about the fiber core 12, and a coating of gold ultra-thin film 16.

In this embodiment, cladding 14 may comprise polymethyl methacrylate (PMMA), which is a synthetic polymer, commonly known as acrylic or Plexiglas. PMMA is a transparent thermoplastic often used as a lightweight and shatter-resistant alternative to glass. In certain embodiments, the cladding 14 may include a doubled cladding structure having a first cladding having 5 micrometers to about 20 micrometers radius of a first material and a second cladding having a radius from 20 micrometers to about 62.5 micrometers of a second material. It should be noted that the first and second claddings provide a waveguide structure for light propagation along the fiber core 12 with negligible transmission loss even at elevated temperatures or in high radioactive environments.

Further, the fiber core 12 includes a series of grating elements 18 that are configured to reflect in phase wavelengths of light corresponding to a grating period of the grating elements 18. In this exemplary embodiment, the fiber core 12 includes PMMA (or other materials such as silica, glass, etc.) and the grating elements 18.

The grating structure 18 is formed through high-power and phase mask inscribing technique followed by a thermal post-treatment process. In operation, structure 20 is inscribed on the fiber core 12 through the high-power and phase mask inscribing technique. In this exemplary embodiment, structure 20 is inscribed on the fiber core 12 using fast and local melting and quenching processes such that an elasto-optical coefficient of the fiber optic sensor 10 is independent of any cluster morphology. In addition, the high-power laser pulse induces an effective refractive index modulation of an order of about 0.2-0.5% in the fiber core 12.

Further, under-constrained structure 22 composed of distorted short-range and dangling bonds is converted into a compact over-constrained rigid tetrahedral structure through a thermal post-treatment process. In this exemplary embodiment, the thickness of grating blocks of the under-constrained and over-constrained structure is about 0.25 micrometers along the fiber axis and has a diameter of about 9 micrometers. The alternating modulated microcrystalline and over-constrained structure blocks effectively form a mass density modulation that introduces an effective refractive index modulation as being a Bragg reflector for constructive light interference at each block interface. The conversion of the under-constrained structure 22 to the over-constrained tetrahedral structure is achieved through isothermal and isochronal processes.

Beneficially, the transformation to a PMMA structure facilitates operation of the fiber optic sensor 10 at elevated temperatures and radiation. Further, the modification of the fiber material microstructure through the isothermal process reduces a grain size distribution and a grain boundary density in the microcrystalline grating structure 18. The reduction in the grain size distribution substantially reduces a residual strain between the fiber core 12 and cladding interface 14 thereby enhancing the transmission properties of the fiber optic sensor 10.

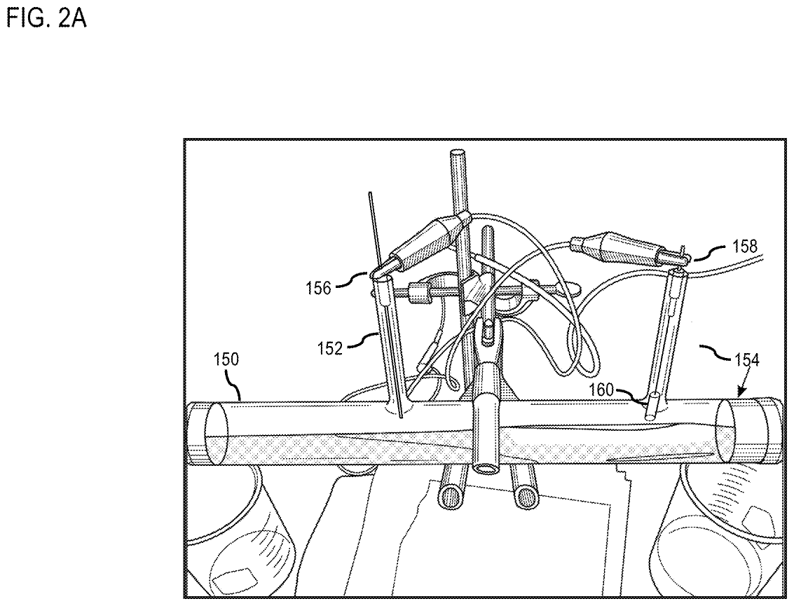

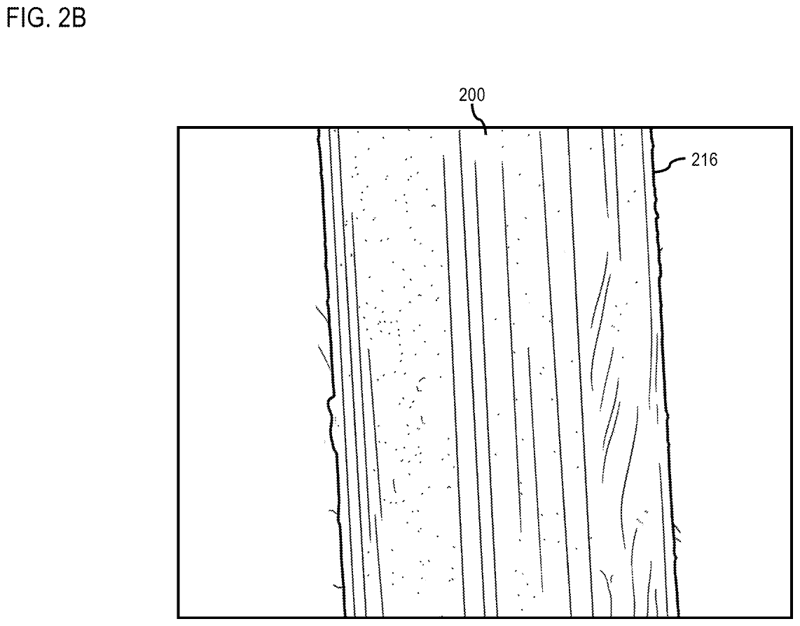

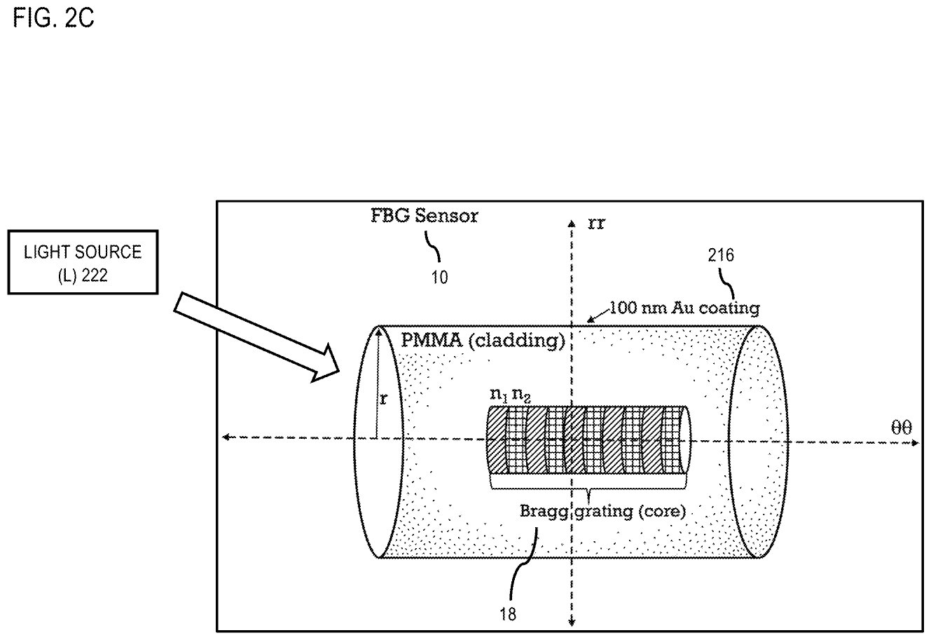

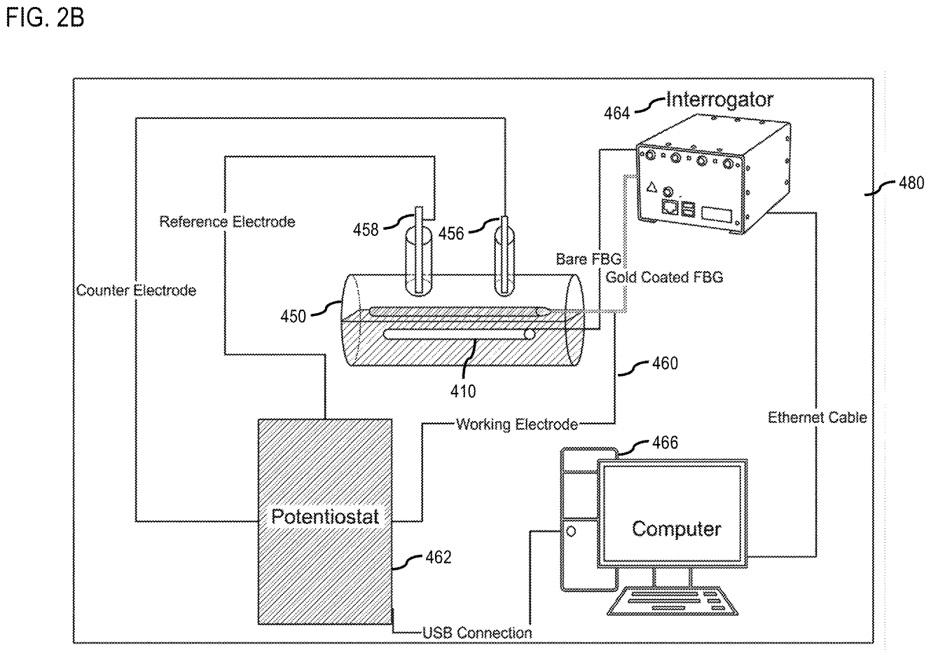

During operation, an input broadband light signal 43 is provided to the fiber optic sensor 10 by the light source L 222 (see FIG. 2C) and a portion of the input broadband light signal 26 is reflected by a respective grating element 18 in phase and corresponding to certain wavelengths of light, while remaining wavelengths are transmitted as represented by a transmitted signal 28. The reflection spectrum generated from the grating elements 18 facilitates simultaneous detection of plurality of parameters of the environment. Examples of such parameters include dynamic strain, radiation, vibration, and temperature, as those skilled in the art will plainly recognize. Further, the periodic or quasiperiodic modulated microcrystalline and rigid tetrahedral grating structure 14 described above reduces the thermo-optical effect fluctuation in the fiber optic sensor 10 thereby enabling detection of multiple parameters in high temperature environments with highly thermal stability. In one exemplary embodiment, fiber optic sensor 10 is operable to detect a plurality of parameters in an environment having a temperature of about 800 °C. The grating structure 14 and packaging material of the fiber optic sensor 10 described above may be selected based upon measurement of desired parameters of an environment.

Referring now to FIG. 2A, a view showing an electrochemical cell 150, in accordance with an embodiment of the present disclosure. The electrochemical cell 150 had an elongated tubular shape with diameter of 2.5 cm and length 25 cm. The cell volume was 125 mL. On the top of the cell, a first vertical port 152 and a second vertical port 154 are provided for a counter electrode 156, a reference electrode 158 and solution management were made having 5 cm length and 0.5 cm in diameter. The sensing or working electrode 160 was PMMA fiber having a 125 μm diameter.

Referring also to FIG. 2B which is an electron micrograph of a fiber optic sensor 200, fiber optic sensor 200 was coated with 100 nm Au layer 216.

Referring again to FIG. 2A, sensing or working electrode 160 was suspended horizontally along the cell 150 interior and immersed into solution S. The connection of the sensing electrode 160 to the potentiostat 462 (see FIG. 2D) was made outside of the cell using specially designed gold foil wrap. The entire length of the sensing electrode 160 was 50 cm and one of its ends was connected to the interrogator 464 (see FIG. 2D). Prior to measurements, after adding the Cl-containing aliquot to the solution in the cell, a 20-minute pause was made to ensure that the Cl-content is uniformly distributed by diffusion process. As will be described below, the performance of the sensing electrode 160 for each solution was tested at constant temperature and pressure. Sensing measurements were conducted at five different potentials: 200 mV; 300 mV; 400 mV; 500 mV and 600 mV vs. Ag/AgCl reference electrode. For each potential, an average of ten consecutive readings were taken to obtain the result.

In yet another aspect, provided is a method for sensing a chemical species in a solution. The method includes obtaining an electrochemical cell having an elongated tubular shape for receiving a solution of interest; positioning an optical probe within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding; and providing an input broadband light signal to the optical sensor.

In some embodiments, the cladding of the optical probe comprises polymethyl methacrylate (PMMA) or other materials such as silica, glass, etc.

In some embodiments, the sensing mechanism of the optical probe combines strain dependence of the Brag wavelength with the change in capillary pressure on the fiber induced by a species adsorption process.

In some embodiments, the chemical species is chloride ions.

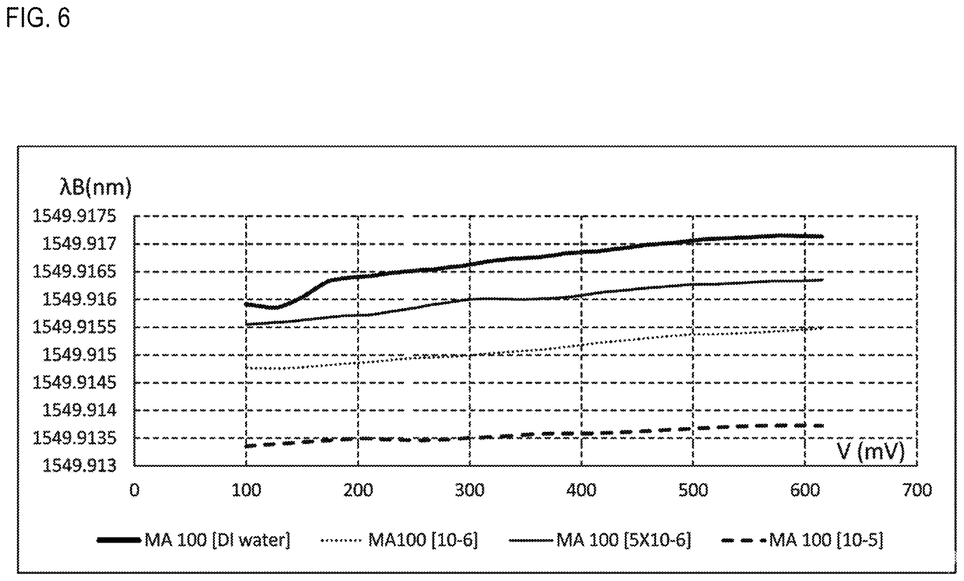

FIG. 6 shows the effect of electrocapillarity on wavelength, according to an embodiment of the present disclosure. As may be seen, four separate graphs were developed, representing four different levels of MA 100. The electrocapillarity effect on the wavelength was measured while increasing the voltage in each case.

FBG sensors 10 may utilize a number of optically transparent materials for their fabrication. As is known, they operate as optical probes for accurate measurements of strain, pressure, stress, vibrations, and temperature. As shown in FIG. 1 and FIG. 2C, the Fiber Bragg Grating 18 represents periodically spaced regions in the fiber core altered to have different refractive indexes. If the wavelength of the input light meets the brag grating condition, it is reflected without attenuation of its amplitude. This wavelength is called resonant wavelength or the Bragg wavelength, and it is a function of the grating periodicity and effective refractive index of the grating. The Bragg wavelength of the FBGs is very sensitive to the temperature change and longitudinal strain in the fiber. The shift in the Bragg wavelength (Δλ_{B}) is used as the sensing signal and it is defined as:

(Δλ_{B})/λ_{B}=(1−Pₑ)ε+(α_{Λ}∓αₙ)ΔT (1)

Here, ε is the longitudinal strain in the grating and ΔT is temperature change. The Pₑ is the strain-optic coefficient while as and an, are the thermal expansion coefficient and the thermo-optic coefficient, respectively.

In this example case, the portion of the fiber with Bragg grating was 1.8 cm. The entire fiber including the part with the FBG coated with 100 nm Au thin film layer by sputtering. This converted the FBG to a conductive working/sensing electrode 160 with surface having chemical preference for Cl⁻ adsorption (see FIG. 2B). The parameters of the FBG sensor used for data analysis in the further text are summarized in Table 1.

TABLE 1
Parameters of FBG Electrode

| Parameter | Value | |
|---|---|---|
| E | 3.0 × 10⁹ | Pa |
| ν | 0.4 | |
| d | 125 × 10⁻⁶ | m |
| Pₑ | 0.28 | |
| λ_{B} | 1565 × 10⁻⁹ | m |

### Examples

### Apparatus

A schematic of apparatus 480 is shown in FIG. 2D. As shown, apparatus 480 consists of a potentiostat 462 which provides electrochemical potential control on the working/sensing FBG electrode 460, the electrochemical cell 450 with reference electrode 458 (Ag/AgCl) and counter electrode 456 (Pt), an optical probe 410, interrogator unit (Smart Scope) 464 and computer 466. The optical probe 410 (FBG with PMMA surface) was also located in cell 450 (see FIG. 2A). Its signal was subtracted from the signal of the sensing FBG electrode 460 (gold coated) to implement a differential sensing concept. This helped to remove the noise and improve SNR. The interrogator unit 464 and potentiostat 462 were connected to computer 466 which enabled simultaneous and synchronized data acquisitions with 5 Hz (interrogator) and 10 Hz frequency (potentiostat).

### General Details

Six solutions were used to test sensor performance. They were made using 0.1 M HClO₄ as a background electrolyte and having concentrations of Cl⁻ ions ranging from 0 M to 10⁻³ M. All solutions were prepared with ultra-pure water (18.2 MΩ) and analytical grade chemicals (NaCl, HClO₄) purchased from Sigma Aldrich. Experiments were conducted in a sequence starting from 0 M Cl⁻ solution and, for each subsequent experiment, the concentration of Cl⁻ is increased by adding appropriate aliquoted of 0.1 M HClO_{4+0.1} M NaCl.

Referring again to FIG. 2A, the electrochemical cell 150 had an elongated tubular shape with a diameter of 2.5 cm and length 25 cm. The cell 150's volume was 125 mL. On the top of the cell 150, vertical ports for the counter electrode 156, reference electrode 158 and solution management (not shown) were made having 5 cm length and 0.5 cm in diameter. The sensing/working electrode was PMMA fiber having 125 μm diameter. It was coated with a 100 nm Au layer, (see FIG. 2B). Such sensing/working electrode 160 was suspended horizontally along the cell interior and immersed into solution. The connection of the sensing electrode 160 to the potentiostat 462 (see FIG. 2D) was made outside of the cell using specially designed gold foil wrap. The entire length of the sensing electrode 160 was 50 cm and one of its ends was connected to the interrogator 464 (see FIG. 2D). Prior to measurements, after adding the Cl⁻ containing aliquot to the solution in the cell 150, a 20-minute pause was made to ensure that the Cl⁻ content is uniformly distributed by diffusion process. Performance of the sensing electrode 160 for each solution is tested at constant temperature and pressure. Sensing measurements were conducted at five different potentials: 200 mV; 300 mV; 400 mV; 500 mV and 600 mV vs. Ag/AgCl reference electrode 158. For each potential, an average of ten consecutive readings are taken as the result.

### Results and Discussion

Chloride adsorption on noble metal electrodes has been the subject of many studies over the years. In particular, the Cl-adsorption on polycrystalline and single crystal gold electrodes was investigated using a variety of different techniques. In general, these reports are divergent in some details, but they do agree on common results; the coverage and the strength of chloride adsorption increases with more positive potential and higher concentration of Cl⁻. The potential region of interest for sensor performance is the double layer region of Au where Cl⁻ forms a disordered phase whose coverage and density increases with more positive potentials.

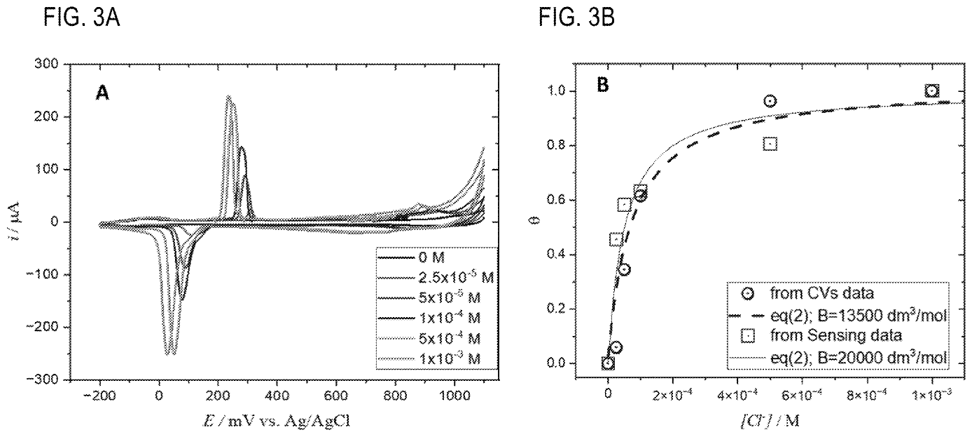

Referring now to FIG. 3A, to identify the optimum potential range for sensor operation, a set of cyclic voltammograms (CVs) were performed on Au coated fiber electrode in solutions with 0.1 MHClO₄+X M [Cl⁻] (X=0, 2.5×10⁻⁵, 5×10⁻⁵, 10⁻⁴, 5×10⁻⁴ and 10⁻³), FIG. 2A. The focus of testing was the potential region where adsorption/desorption of disordered Cl phase occurs (−100 mV<E<600 mV). This is evident by the existence of the positive and negative peaks whose area and position show strong dependence on the Cl-concentration in the electrolyte. The adsorption/desorption peak shift ~35 mV in more negative/positive direction per one decade increase in [Cl⁻]. This value is close to the previously reported one for Au(111) 11 and suggests that an FBG electrode has a strong interaction with absorbate.

After inspecting the anodic part of CVs in FIG. 3, it is observed that the main Cl-adsorption peaks are confined between the 200 mV and 400 mV potentials. A further potential sweep in a more positive direction does not reveal any noticeable peaks until 900 mV where Cl adlayer ordering occurs and the onset of Cl chemisorption. Referring again to FIG. 3A, the corresponding negative peak occurring during cathodic sweep shows at ~700 mV. Considering that Cl chemisorption on an Au surface leads to formation of an AuCl compound and anodic dissolution of Au, it can be said that the potential range for sensor operation is between the range of 200 mV and 600 mV. In this range, the Au surface stays unaltered and only physiosorbed Cl adlayer forms. By performing the integration of the anodic peak for each curve/electrolyte between 100 mV and 400 mV limits, the physiosorbed Cl adlayer may be characterized. These results serve to construct the adsorption isotherm after the data are normalized by the area of the largest peak obtained from the 10⁻³ M [Cl⁻] electrolyte. These data are plotted in FIG. 3B. The observed θ vs. [Cl⁻] trend is modeled by the Langmuir isotherm (see equation (2)).

θ=(B·Cl⁻)/(1+B·Cl⁻) (2)

Here, the term B represents the equilibrium adsorption constant. Referring to FIG. 3B, the model (dotted line) fit to experimental data yields the R²>0.95, which confirms that Langmuir adsorption formalism can be employed in further phenomenological description of the sensing process.

### Phenomenological Description of Cl⁻ Sensing

The sensing phenomenon starts with the re-statement of the Gibbs adsorption isotherm for single solute-solution system and dilute solution approximation. For constant pressure, temperature, and potential it states as:

Γ_{Cl}=−[Cl⁻]/RT·∂γ/∂[_{Cl-}]|_{(T,p,E=const)} (3)

The term Γ_{Cl} represents the surface concentration of adsorbed Cl adlayer [mol·m⁻²] and γ is the surface energy [J·m⁻²]. The other terms are as defined previously. Γ_{Cl} can be expressed in terms of its coverage, and the maximum concentration of the Cl adlayer at given conditions, Γₘₐₓ, as:

Γ_{Cl}=θ·Γₘₐₓ. (4)

Combining eqs (3-4) with eq (2) provides a differential form of the thermodynamic model which describes the decrease in the surface energy due to Cl-adsorption process and change in concentration of Cl⁻ ions in the solution.

B/(1+B[C⁻])∂[Cl⁻]=−1/(ΓₘₐₓRT)∂γ·−|_{(p,T,E=const)} (5)

After integration of the above equation in the limits of [Cl⁻]=0; γ=γ_{Au} and [Cl⁻]=[Cl⁻]; γ=γ_{Cl/Au}. The surface energy change as a function of [Cl⁻] is given by:

γ_{(Cl/Au)}=γ_{Au}−RT Γₘₐₓ ln(1+B[Cl⁻] (6)

Here, γ_{Cl/Au} is the surface energy of the Au electrode with the adsorbed Cl adlayer of arbitrary coverage. The above expression is an integral restatement of eq (3) and serves as the starting point in development of the phenomenological model for a sensing process.

Next, the capillary pressure on the FBG electrode is defined. For cylindrical geometry of the fiber with diameter d, the capillary pressure is:

Δp=2γ/d (7)

The capillary pressure is compressive, and thus, according to the co-ordinate system adopted in for FIG. 2C, it can be expressed in terms of the stress in the fiber operating in radial (σrr) and/or longitudinal directions (σ_{θθ}) as:

Δp=−σᵣᵣ=(1/v)σ_{θθ}=2γ/d (8)

Here v is Poisson's ratio for the fiber material. Furthermore, the stress in the fiber in the longitudinal direction can be re-written in terms of longitudinal strain in the fiber (σ_{θθ}) and Young's modules of the fiber material as:

σ_{θθ}=Eε_{θθ} (9)

Combining eqs (8-9) reveals the expression defining the longitudinal strain in the fiber as a function of the surface energy:

ε_{θθ}=(2v/(E·d))γ (10)

In the case of the gold coated FBG electrode in solution with Cl⁻ ions, the surface energy of is dependent on [Cl⁻] (eq (6)), and the above expression transforms to:

ε_{θθ}=(2v/(E·d){γ_{Au}−RT·Γₘₐₓ·ln(1+B[Cl⁻)}. (11)

The eq (11) represents the essence of the sensing mechanism. It describes a phenomenological relation between the mechanical deformation in the fiber (ε_{θθ}) exerted by the capillary pressure dependent on Cl⁻ concentration in the solution. The link between these two is the surface energy decrease due to adsorption process.

### Transducer Equation

As discussed, the Examples implement differential sensing approaches. There are two FBGs involved: the sensing fiber with gold coated surface and the referent one with PMMA surface. The first one provides a sensing signal, which is dependent on [Cl⁻] and potential while the second one provides the referent signal. For co-ordinate system adopted here, FIG. 2C, and T=const. eq (1) transforms to:

(Δλ_{B})/λ_{B}=(1−Pₑ)ε_{θθ}. (12)

In the case of sensing fiber, combining eqs (11 and 12) it follows:

(Δλ_{B})₁/λ_{B}=(1−Pₑ)(2v/(E·d))·{γ_{Au}−RTΓₘₐₓ·ln(1+B[Cl⁻])} (13)

In the case of the referent fiber, combining eqs (10 and 12) provides:

(Δλ_{B})₂/λ_{B}=(1−Pₑ)(2v/(E·d))γ_{PMMA}. (14)

Subtraction of these two signals leads to the equation that models the behavior of the sensing system:

(Δλ_{B})₁−(Δλ_{B})₂=λ_{B}(1−Pₑ)(2v/(E·d))·{γ_{Au}−γ_{PMMA}−RT·Γₘₐₓ·ln(1+B[Cl⁻])}. (15)

The notation in this equation compresses to: (Δλ_{B})₁−(Δλ_{B})₂=(Δλ_{B})_{Δ}; and λ_{B} (1−Pₑ) 2v/(E·d)·{γ_{Au}−γ_{PMMA}}=Δλ_{B0}; arriving to the final form of the transducer equation which is used to model the experimental data.

(Δλ_{B})_{Δ}=Δλ_{B0}−λ_{B}(1−Pₑ)(2v/(E·d)·RT·Γₘₐₓ·ln(1+B[Cl⁻) (16)

The values of E, v, Pₑ for the PMMA fiber material and fiber diameter are listed in Table 1. Note that the real value of Δλ_{B0} in experiments (case [Cl⁻]=0) is somewhat different from one defined here. The real one also absorbs the Bragg wavelength shift difference related to residual strains in fibers raised during their placement and fixing in the cell and potential bending. It also absorbs any inherent difference in Bragg gratings between two fibers related to manufacturing process. Nevertheless, these effects do not change the mathematical form of eq (16). Their contributions reflect to the integral value of Δλ_{B0} while keeping in mind that its “generality” goes beyond the description presented here.

### Sensing Data

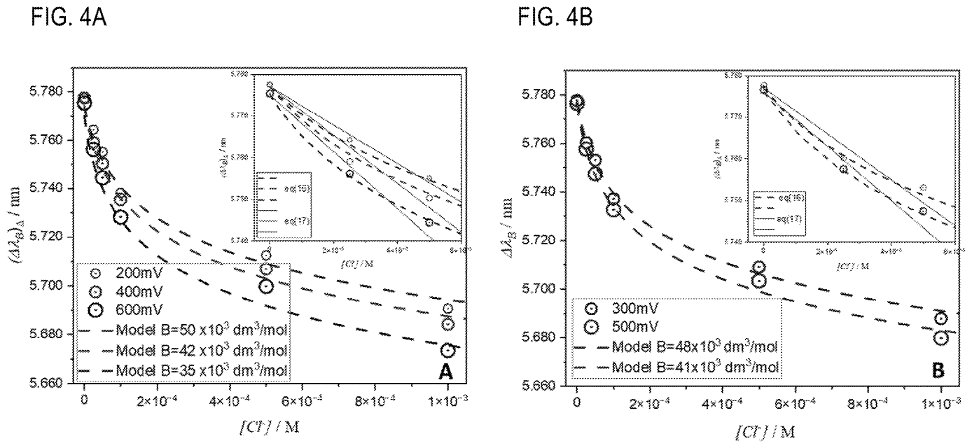

Referring now to FIGS. 4A and 4B, five sets of sensing data are presented. Each set shows our sensor responses to six values of [Cl⁻] ranging from 0 to 10⁻³ M. They are recorded at constant potential of the FBG electrode. FIG. 4A shows data sets recorded at 200 mV, 400 mV and 600 mV while FIG. 4B shows data sets recorded at 300 mV and 500 mV. The dotted lines in each graph are eq (16) fits to experimental data. The insets in each graph show zoom-in the crowded portion of the data for [Cl⁻]<10⁻⁴ M range. As shown, the decrease in the (Δλ_{B})_{Δ} value is a strong function of the increasing chloride concentration in the solution. The observed trend is well described by our transducer equation which validates the idea for sensing approach and its description.

The relative change of the signal is larger at more positive potentials. This is not surprising as more adsorbed chloride is present on the Au surface at higher potentials. Sensor behavior is not linear with [Cl⁻]. This is already suggested by mathematical form of eq (16). Nevertheless, the concentration range covered in the experiments was quite large, and probably not of interest for true applications. Focusing on detecting traces of Cl⁻ in the solution (see insets in FIGS. 4A and 4B), a quasi-linear behavior can be assumed. Assuming a linear approximation of Langmuir adsorption model (θ=B·[Cl⁻) the transducer equation transforms into a linear relationship in terms of [Cl⁻];

(Δλ_{B})_{Δ}=Δλ_{B0}−λ_{B}(1−Pₑ)(2v/(E·d))·RT·Γₘₐₓ·B[Cl⁻] (17)

The fits of the linear model for low concentration range are shown in FIGS. 4A and 4B, as solid lines.

Inspections of the model and experimental data suggest that the relative sensitivity of the system, (d((Δλ_{B})_{Δ}))/(d([Cl⁻])), is dependent on concentration. This is illustrated by using two limiting cases. The first one is for [Cl⁻]→0; where relative sensitivity equals to:

(d(Δλ_{B})_{Δ}))/(d([Cl⁻])|_{[Cl-]→0)}=B·λ_{B}(1−Pₑ)2v/(E·d)·RT·Γₘₐₓ (18)

The second one is for [Cl⁻]→∞. In a practical sense, this limit is set by chloride salt solubility ([Cl⁻]*=((Kₚ/Mⁿ⁺))^{1/n}). Therefore, the relative sensitivity is given as:

((d((Δλ_{B})_{Δ}))/(d([Cl⁻])))|_{([Cl-]→∞)}=1/[Cl⁻]*·λ_{B}(1−Pₑ)2v/(E·d)·RT·Γₘₐₓ (19)

For any soluble chloride salt, B>>1/[Cl⁻]* inequality always holds and comparison of eq (18) and eq (19) shows that our sensor performs better in solutions with a low chloride concentration. However, the absolute sensitivity limit of our system is set by the quality of the interrogator. In this case, it had a detection limit of 0.5 μm for Δλ_{B} shift which allows detection of [Cl⁻] below 10⁻⁶ M for our sensor/fiber design. The 1/d dependence of the relative sensitivity does suggest that further improvements of the sensor performance can be achieved by using a fiber with smaller diameter. Nevertheless, this can be beneficial only if the fiber integrity and handling is not jeopardized.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

In the event that any patents, patent applications, or other references are incorporated by reference herein and define a term in a manner or are otherwise inconsistent with either the non-incorporated portion of the present disclosure or with any of the other incorporated references, the non-incorporated portion of the present disclosure shall control, and the term or incorporated disclosure therein shall only control with respect to the reference in which the term is defined and/or the incorporated disclosure was originally present.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

Illustrative, non-exclusive examples of systems and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

A1. An optical probe for sensing a chemical species in a solution, comprising: a fiber core having a plurality of Bragg grating elements, the grating elements comprise periodically spaced regions in the fiber core altered to have different refractive indexes; a cladding disposed about the fiber core; and a gold ultra-thin film coating disposed about the cladding.

A2. The optical probe of paragraph A1, wherein the cladding comprises polymethyl methacrylate (PMMA), silica, or glass.

A3. The optical probe of paragraphs A1 or A2, wherein the cladding comprises a double cladding structure having a first cladding having 5 micrometers to about 20 micrometers radius of a first material and a second cladding having a radius from 20 micrometers to about 62.5 micrometers of a second material.

A4. The optical probe of paragraph A3, wherein the first and second claddings provide a waveguide structure for light propagation along the fiber core with negligible transmission loss even at elevated temperatures or in high radioactive environments.

A5. The optical probe of any of paragraphs A1-A4, wherein the plurality of Bragg grating elements are configured to reflect in phase wavelengths of light corresponding to a grating period of the grating elements.

A6. The optical probe of any of paragraphs A1-A5, wherein the sensing mechanism combines strain dependence of the Brag wavelength with the change in capillary pressure on the fiber induced by a species adsorption process.

A7. The optical probe of any of paragraphs A1-A6, wherein the chemical species is a chloride ion.

B1. An apparatus for sensing a chemical species in a solution, comprising: an electrochemical cell having an elongated tubular shape for receiving a solution of interest; an optical probe positioned within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding.

B2. The apparatus of paragraph B1, further comprising a first vertical port for receiving a reference electrode therethrough.

B3. The apparatus of paragraph B1 or B2, further comprising a second vertical port for receiving a counter electrode therethrough.

B4. The apparatus of paragraph B3, further comprising a sensing or working electrode disposed within the apparatus.

B5. The apparatus of any of paragraphs B1-B4, further comprising a light source for providing an input broadband light signal to the optical sensor.

B6. The apparatus of any of paragraphs B1-B5, wherein the cladding of the optical probe comprises polymethyl methacrylate (PMMA), silica, or glass.

B7. The apparatus of any of paragraphs B1-B6, wherein the plurality of Bragg grating elements of the optical probe are configured to reflect in phase wavelengths of light corresponding to a grating period of the grating elements.

B8. The apparatus of any of paragraphs B1-B7, wherein the sensing mechanism of the optical probe combines strain dependence of the Brag wavelength with the change in capillary pressure on the fiber induced by a species adsorption process.

B9. The apparatus of any of paragraphs B1-B8, wherein the chemical species is a chloride ion.

C1. A method for sensing a chemical species in a solution, comprising: obtaining an electrochemical cell having an elongated tubular shape for receiving a solution of interest; positioning an optical probe within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding; and providing an input broadband light signal to the optical sensor.

C2. The method of paragraph C1, wherein the cladding of the optical probe comprises polymethyl methacrylate (PMMA), silica, or glass.

C3. The method of paragraph C1 or C2, wherein the sensing mechanism of the optical probe combines strain dependence of the Brag wavelength with the change in capillary pressure on the fiber induced by a species adsorption process.

C4. The method of any of paragraphs C1-C3, wherein the chemical species is a chloride ion.

### INDUSTRIAL APPLICABILITY

The optical probe and method disclosed herein are applicable to the corrosion, biomedical, chemical, health and oil and gas industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. An optical probe for sensing a chemical species in a solution, comprising:
a fiber core having a plurality of Bragg grating elements, the grating elements comprise periodically spaced regions in the fiber core altered to have different refractive indexes;
a cladding disposed about the fiber core; and
a gold ultra-thin film coating disposed about the cladding.

2. The optical probe of claim 1, wherein the cladding comprises polymethyl methacrylate (PMMA), silica, or glass.

3. The optical probe of claim 1, wherein the cladding comprises a double cladding structure having a first cladding having 5 micrometers to about 20 micrometers radius of a first material and a second cladding having a radius from 20 micrometers to about 62.5 micrometers of a second material.

4. The optical probe of claim 3, wherein the first and second claddings provide a waveguide structure for light propagation along the fiber core with negligible transmission loss even at elevated temperatures or in high radioactive environments.

5. The optical probe of claim 1, wherein the plurality of Bragg grating elements are configured to reflect in phase wavelengths of light corresponding to a grating period of the grating elements.

6. The optical probe of claim 1, wherein the sensing mechanism combines strain dependence of the Brag wavelength with the change in capillary pressure on the fiber induced by a species adsorption process.

7. The optical probe of claim 1, wherein the chemical species is a chloride ion.

8. An apparatus for sensing a chemical species in a solution, comprising:
an electrochemical cell having an elongated tubular shape for receiving a solution of interest;
an optical probe positioned within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding.

9. The apparatus of claim 8, further comprising a first vertical port for receiving a reference electrode therethrough.

10. The apparatus of claim 9, further comprising a second vertical port for receiving a counter electrode therethrough.

11. The apparatus of claim 10, further comprising a sensing or working electrode disposed within the apparatus.

12. The apparatus of claim 10, further comprising a light source for providing an input broadband light signal to the optical sensor.

13. The apparatus of claim 8, wherein the cladding of the optical probe comprises polymethyl methacrylate (PMMA), silica, or glass.

14. The apparatus of claim 8, wherein the plurality of Bragg grating elements of the optical probe are configured to reflect in phase wavelengths of light corresponding to a grating period of the grating elements.

15. The apparatus of claim 8, wherein the sensing mechanism of the optical probe combines strain dependence of the Brag wavelength with the change in capillary pressure on the fiber induced by a species adsorption process.

16. The apparatus of claim 8, wherein the chemical species is a chloride ion.

17. A method for sensing a chemical species in a solution, comprising:
obtaining an electrochemical cell having an elongated tubular shape for receiving a solution of interest;
positioning an optical probe within the electrochemical cell, the optical probe comprising a fiber core having a plurality of Bragg grating elements, the grating elements comprise periodically spaced regions in the fiber core altered to have different refractive indexes; a cladding disposed about the fiber core; and a gold ultra-thin film coating disposed about the cladding; and
providing an input broadband light signal to the optical sensor.

18. The method of claim 17, wherein the cladding of the optical probe comprises polymethyl methacrylate (PMMA), silica, or glass.

19. The method of claim 17, wherein the sensing mechanism of the optical probe combines strain dependence of the Brag wavelength with the change in capillary pressure on the fiber induced by a species adsorption process.

20. The method of claim 17, wherein the chemical species is a chloride ion.
